(19) United States
(12) Patent Application Publication
(10) Pub. No.: US 2023/0182055 A1
(43) Pub. Date: Jun. 15, 2023

(54) PURIFIER

(71) Applicant: Airplove (Xiamen) Electronic Co., Ltd., Xiamen (CN)

(72) Inventors: Jueyuan Chen, Xiamen (CN); Lingfeng Lin, Xiamen (CN); Yujia Guo, Xiamen (CN); Tingping Lei, Xiamen (CN)

(21) Appl. No.: 17/648,445

(22) Filed: Jan. 20, 2022

(30) Foreign Application Priority Data
Dec. 13, 2021 (CN) 202123113823.X

(51) Int. Cl. B01D 46/00; B01D 46/24; B01D 46/42; A61L 9/20

(52) U.S. Cl. CPC B01D 46/0028; B01D 46/2403; B01D 46/4227; A61L 9/20; B01D 2265/028; B01D 2279/65; A61L 2209/14; A61L 2209/12; A61L 2209/15

(57) ABSTRACT

The present invention discloses a purifier, including a purifier body and a filter unit arranged in a purification chamber of the purifier body. A side wall of the purification chamber has an opening to which a cover is mounted. The filter unit is an integrated filter and disinfection body, which includes a filter body and a disinfection body which are detachable from each other. The filter unit is mounted inside the purification chamber and electrically connected to the purifier body to supply power to the disinfection body. According to the arrangement of the filter and disinfection body in the present invention, the disinfection body and the filter body are collectively designed into one integrated structure, so that the disinfection body and the filter body can be mounted inside or removed from the purifier body as a whole, which is convenient and fast. In addition, the disinfection body and the filter body in the present invention are assembled in a detachable manner, so that the disinfection body and the filter body can be replaced, repaired and maintained separately, and the long-term use costs of users are greatly reduced, thereby solving the technical problem existing in the related art.

### TECHNICAL FIELD

The present invention relates to the field of air purifiers, and in particular to a purifier which can realize the convenient replacement of a filter body and in which filtering and disinfection functions are integrated.

### BACKGROUND

The quality of indoor air is very important, and air purifiers become more and more popular. The air purifiers have gradually become the necessities of life. They can adsorb, decompose or transform various air pollutants and effectively improve air cleanliness. The pollutants in the air are typically treated by the existing air purifiers by means of filtration, adsorption, etc., but bacteria in the air, or bacteria reproducing inside the machine, especially on a filter screen, after using for a certain period of time, are likely to cause secondary pollution of the purified air.

The mature technologies for sterilization and disinfection of filter structures mainly include: high-temperature sterilization and ultraviolet sterilization. The treatment method of high-temperature sterilization has the problems of large components and high prices. Ultraviolet lamps themselves are low-cost, can destroy the molecular structure of a microbial cell and cause cell death by means of emitted ultraviolet rays to achieve the sterilization effect, and are thus widely used today.

However, in the related art, the filter structure with a disinfection function is either a fixed integrated structure or a separate split structure. The fixed integrated structure can be mounted and dismounted in one step, but has some disadvantages such as being unable to realize separate replacement, maintenance, and repair, and having high use costs. The separate split structure needs to be mounted in multiple steps but can realize separate replacement, maintenance and repair, and has reduced use costs.

Therefore, how to realize the one-step dismounting and mounting of the filter structure with a disinfection function while reducing the use costs to achieve the purpose of convenient replacement, maintenance and repair is one of the technical problems to be solved by technicians in the field of purifiers.

### SUMMARY

To solve the above technical problem, the present invention aims to provide a purifier which can realize the convenient replacement of a filter body and in which filtering and disinfection functions are integrated.

To achieve the above purpose, the present invention uses the following, technical scheme:

a purifier, including a purifier body and a filter unit arranged in a purification chamber of the purifier body.

A side wall of the purification chamber has an opening to which a cover is mounted.

The filter unit is an integrated filter and disinfection body, which includes a filter body and a disinfection body which are detachable from each other.

The filter unit is mounted inside the purification chamber and electrically connected to the purifier body to supply power to the disinfection body.

Further and optionally, the filter unit is cylindrical, and an upper end of the filter unit has an opening as a purified air outlet, which is in communication with an air discharge passage of the purifier body; and

the disinfection body and the filter body are arranged one inside the other, and the filter body is arranged close to and in communication with an air intake passage of the purifier body.

Further and optionally, the disinfection body includes an ultraviolet disinfection lamp and a mounting seat, where

the ultraviolet disinfection lamp is vertically mounted on the mounting seat, and is electrically connected to a first electrical connector arranged on the mounting seat; and

the mounting seat is a base seat having a bottom surface provided with the first electrical connector, and the first electrical connector is electrically connected to a second connector arranged on the purifier body.

Further and optionally, the filter body is cylindrical, the disinfection body is placed in the middle of the filter body, an inner side wall of the filter body is provided with at least one rotational snap-fit notch, and an outer side wall of the disinfection body is provided with at least one rotational snap-fit catch; and

the at least one rotational snap-fit catch is snap-fitted with or detached from the at least one rotational snap-fit notch by rotating the disinfection body relative to the filter body.

Further and optionally, there are a plurality of the rotational snap-fit notches, which are located at a lower edge of the filter body and arranged at intervals; and

the number of the rotational snap-fit catches matches the number of the rotational snap-fit notches.

Further and optionally, the filter unit is provided with at least one limiting boss, and the purification chamber is provided with at least one limiting column; and

the at least one limiting boss is mated and connected to the at least one limiting column to form a mounting limiting structure of the filter unit.

Further and optionally, there are four of the limiting bosses, which are divided into two groups each including two limiting bosses, the two groups of limiting bosses are symmetrically arranged, and the two limiting bosses in each group are arranged one above the other; and

there are two of the limiting columns, which match the two groups of limiting bosses.

Further and optionally, the two limiting bosses in each group are respectively located at upper and lower edges of the filter body.

Further and optionally, the filter unit is provided with a handle, which is located at an upper edge of the filter body.

Further and optionally, the side wall of the purification chamber or/and the cover is/are provided with air inlet holes.

After adopting the technical scheme, compared with the related art, the present invention has the following advantages:

1. A filter unit is provided in the present invention, which is an integrated filter and disinfection body, in which a disinfection body and a filter body are collectively designed into one integrated structure, so that the disinfection body and the filter body can be mounted inside or removed from the purifier body as a whole, which is convenient and fast. In addition, with regard to the filter unit in the present invention, the disinfection body and the filter body are assembled in a detachable manner, so that the disinfection body and the filter body can be replaced, repaired and maintained independently of each, other, and the long-tem use costs of users are greatly reduced, thereby solving the technical problems existing in the related art.

2. The filter unit structure of centralized design is, adopted in the present invention, which improves the utilization rate and arrangement rationality of the inner space of the product.

3. In the present invention, the disinfection body is located in the middle of the filter body, so that the disinfection body and the filter body are arranged one inside the other, and a single disinfection structure can irradiate the filter structure entirely, thereby improving the disinfection efficiency and effect.

### BRIEF DESCRIPTION OF DRAWINGS

FIG. 1 is an exploded view of the structure of a purifier in an embodiment of the present invention;

FIG. 2 is a first schematic diagram of the structure of a filter unit in an embodiment of the present invention;

FIG. 3 is a second schematic diagram of the structure of the filter unit in the embodiment of the present invention;

FIG. 4 is an exploded view of the structure of the filter unit in the embodiment of the present invention,

FIG. 5 is a sectional view of the structure of the filter unit in the embodiment of the present invention; and

FIG. 6 is an enlarged view of the structure at part A in FIG. 5.

### DETAILED DESCRIPTION

In order to make the objectives, technical schemes and advantages of the present invention more apparent, the present invention is further described in detail, in conjunction with the accompanying drawings and embodiments. It should be understood that the specific embodiments described here are intended only to explain the present invention and are not intended to limit the present invention.

In the present invention, it should be noted that the terms “up”, “down”, “left”, “right”, “vertical”, “horizontal”, “inside” and “outside” are all based on the orientation or positional relationship shown in the drawings, only for the convenience of describing the present invention and simplifying the description, and do not indicate or imply that the device or element of the present invention must have a specific orientation, so it cannot be understood as limiting the present invention.

### Embodiment

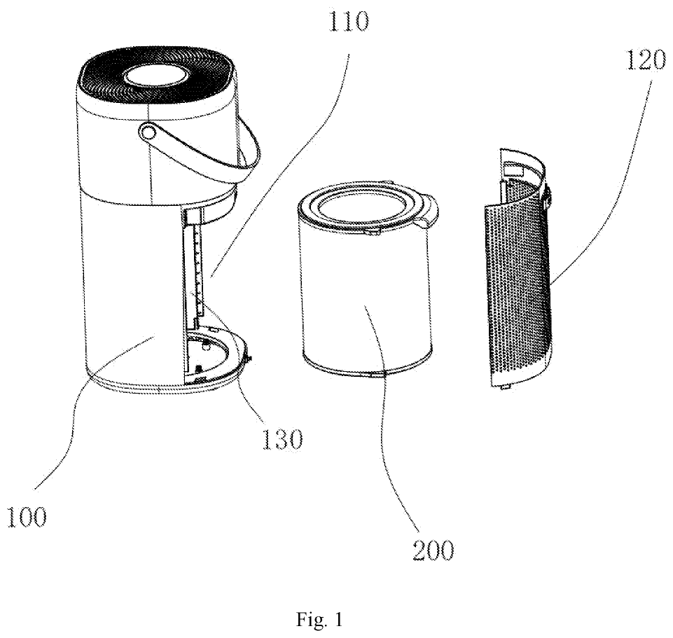

As shown in FIG. 1, the present invention sets forth a purifier, which includes a purifier body 100 and a filter unit 200. The filter unit 200 is an integrated disinfection and purification body, which is mounted into the purifier body 100 from an opening on a side wall of a purification chamber 110 of the purifier body 100 to purify and disinfect air.

As shown in FIG. 1, the purifier body 100 has a purification chamber 110. The purification chamber 110 is located in the lower half of the purifier body 10. The side wall of the purifier body 100 has an opening to form a side opening structure. The opening is in communication with the purification chamber 110. A cover 120 is mounted at the opening. The cover may be connected to the opening by means of snap-fitting, magnetic attraction, hinging, etc., which is a common technology in the field of purifiers and will not be described in detail here.

A plurality of air inlet holes are provided in the side wall of the purifier body 100 corresponding to the purification chamber 110 or in the cover 120 to form an air intake passage, and the purifier body 100 has a purified air discharge passage.

In addition, the purifier body 100 is provided with a second electrical connector (not shown) which can be connected to a power supply, and the second electrical connector may be a plug which can be connected to a power socket to realize the connection between the purifier body and the power supply. Alternatively, the second electrical connector may be a wireless electrical component or a conductor connected to a built-in battery.

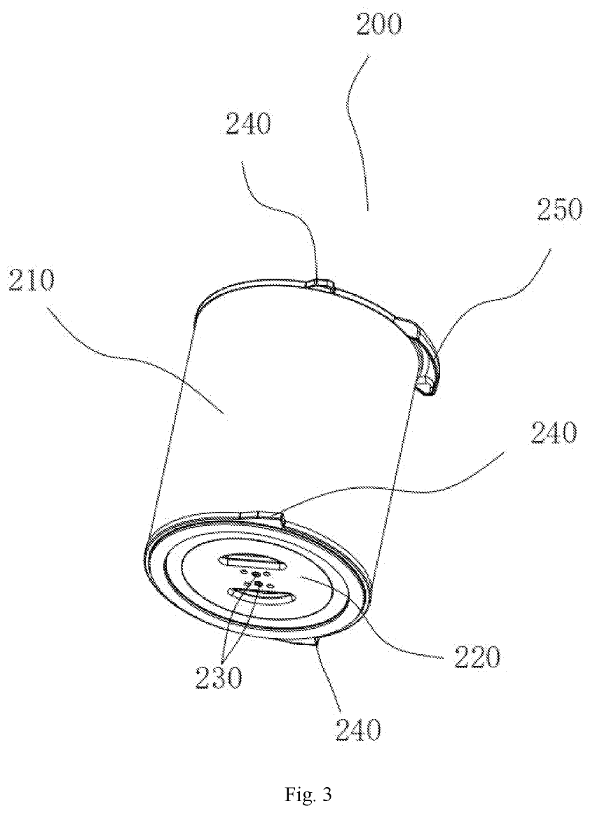

As shown in FIGS. 1 and 3, the filter unit 200 is cylindrical and mounted into the purification chamber 110 from the opening, and the cover 120 is mounted at the opening to realize the installation of the filter unit 200. When the filter unit 200 is mounted inside the purification chamber 110, a first electrical connector 230 provided at the bottom of the filter unit 200 is mated and connected to the second electrical connector to activate the filter unit 200, i.e., to supply power to the filter unit 200. In addition, air enters the air intake passage, is purified by the filter unit 200, and is then discharged from a purified air outlet at the upper end of the purification unit 200 and the air discharge passage of the purifier body 100. The purified air outlet is an opening at the upper end of the purification unit 200, and is in communication with the air discharge passage.

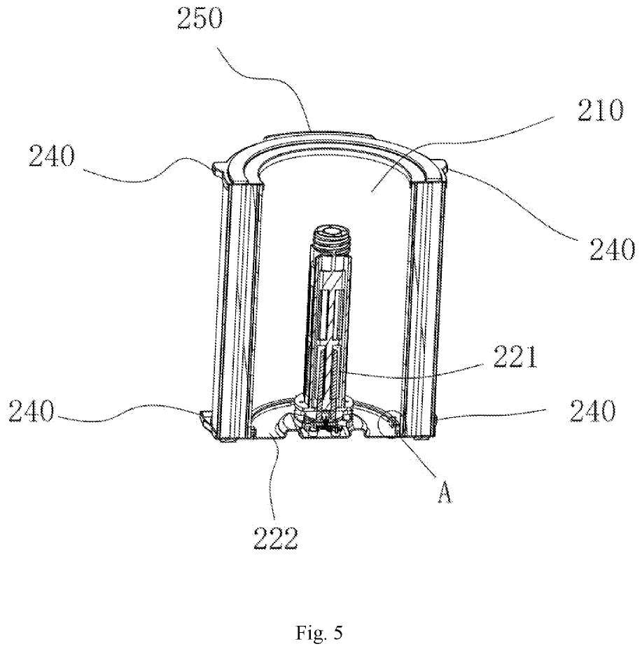

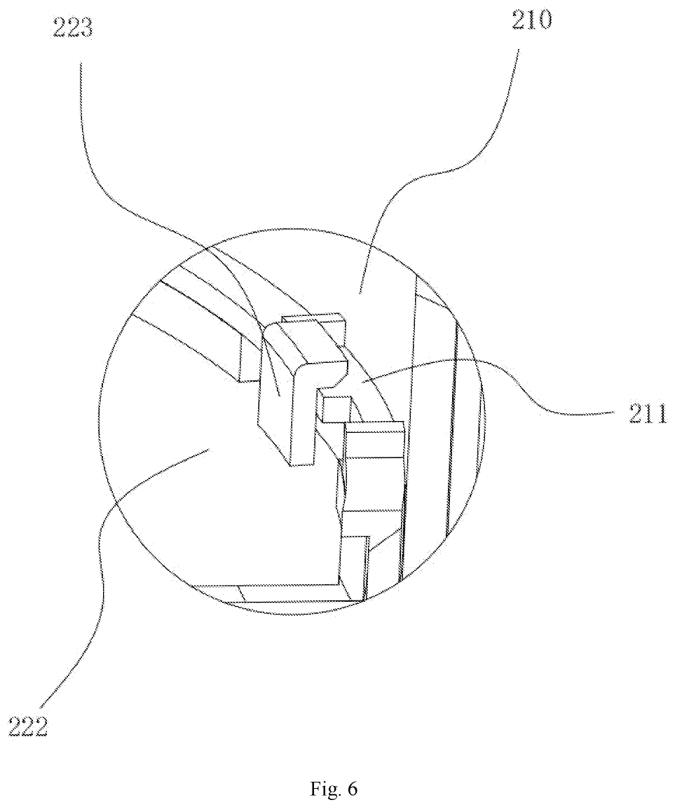

As shown in FIGS. 1-6, the filter unit 200 includes a filter body 210 and a disinfection body 220. The filter body 210 is in an annular shape, the disinfection body 220 is located in the filter body 210, and the filter body 210 is detachably mounted to the disinfection body 220 to form an integrated structure with the two bodies detachable from each other. The disinfection body 220 is provided with a mounting seat 222 on which an ultraviolet disinfection lamp 221 is vertically mounted, and at least one rotational snap-fit notch 211 of the filter body 210 is rotationally snap-fitted with or detached from at least one rotational snap-fit catch 223 on the mounting seat 222, so as to achieve the purpose of replacing the filter body conveniently and improve the maintenance convenience of the purifier.

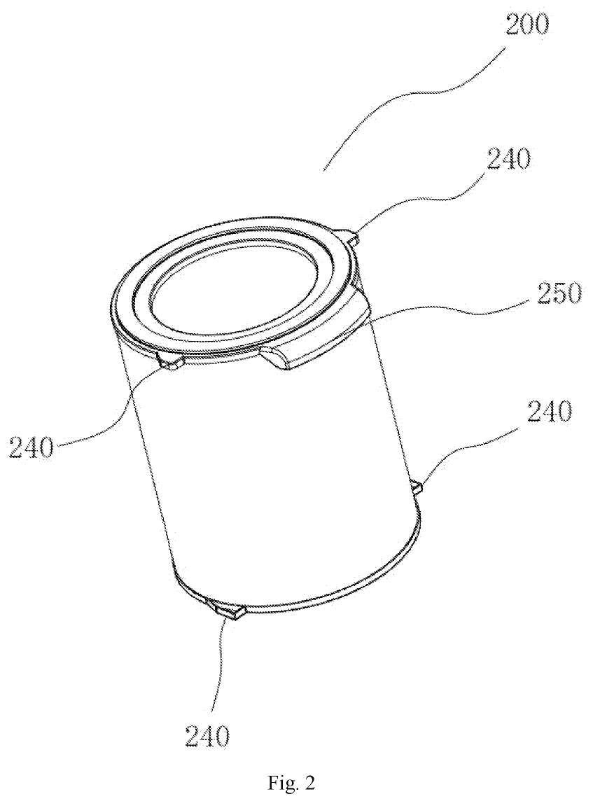

Specifically, as shown in FIGS. 2-6, the disinfection body 220 includes the ultraviolet disinfection lamp 221 and the mounting seat 222. The ultraviolet disinfection lamp 221 is vertically mounted at the center of the mounting seat 22, and a first electrical connector 230 is arranged at the center of a bottom surface of the disinfection body 220, and the first electrical connector 230 is a conductive contact. An inner side wall of the filter body 210 is provided with the at least one rotational snap-fit notch 211, and an outer side wall of the mounting seat 222 is provided with the at least one rotational snap-fit catch 223. In this embodiment, there are a plurality of the rotational snap-fit notches 211 arranged at intervals. The number and positions of rotational snap-fit catches 223 match the number and positions of the rotational snap-fit notches 211.

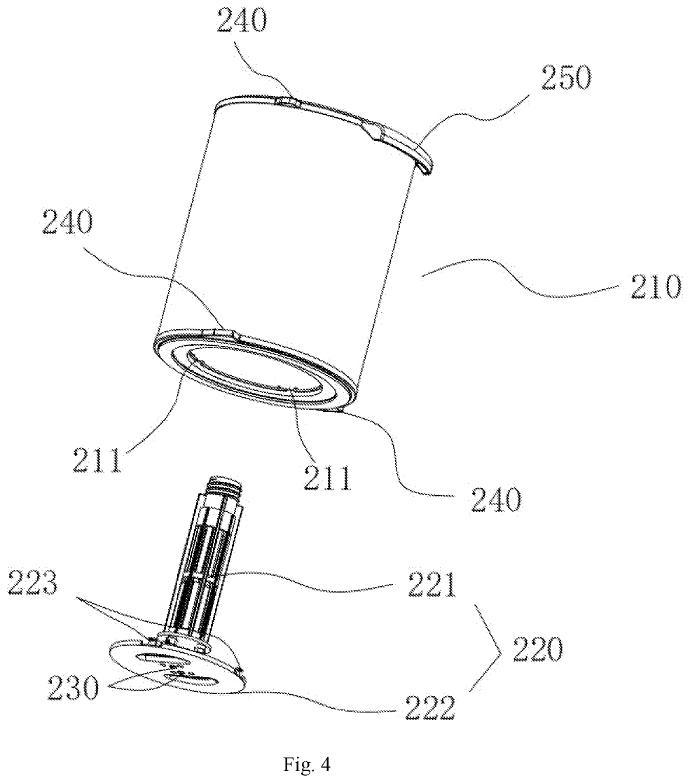

In this embodiment, as shown in FIGS. 4-6, the rotational snap-fit notches 211 are located at a lower edge of the inner side wall of the filter body 210, and the rotational snap-fit catches 223 are located at the outer side wall of the mounting seat 222. There are four rotational snap-fit notches 211 and four rotational snap-fit catches 223, which are all arranged at intervals. The filter body 210 surrounds the outside of the disinfection body 220 and is rotated such that the rotational snap-fit notches 211 are mated and snap-fitted with the rotational snap-fit catches 223 to realize the connection between the disinfection body 220 and the filter body 210. On the contrary, the disinfection body 220 and the filter body 210 are rotated relative to each other such that the rotational snap-fit notches 211 are detached from the rotational snap-fit catches 223, so as to detach the disinfection body 220 from the filter body 210, so that the filter body 210 can be conveniently replaced on a regular basis.

Referring to FIG. 1 and FIGS. 4-6, in order to accurately mount the disinfection and filter body 200 in the purification chamber 110, a mounting limiting structure is arranged between the disinfection and filter body 200 and the purification chamber 110. The mounting limiting structure includes at least one limiting boss 240 and at least one limiting column 130. There are four of the limiting bosses 240, which are divided into two groups each including two limiting bosses, the two groups of limiting bosses 240 are symmetrically arranged, and the two limiting bosses 240 in each group are arranged one above the other. The two limiting bosses 240 in each group are respectively located at upper and lower edges of the filter body 210. The at least one limiting column 130 is an upright column and is mounted in the purification chamber 110. There are two of the limiting columns, which match the two groups of limiting bosses 240. An outer side wall of each limiting boss 240 has a limiting face, which abuts against a side wall of a respective limiting column 130.

As shown in FIGS. 2-6, the filter unit 200 is provided with a handle 250. The handle 250 is located at the upper edge of the filter body 210.

More specifically, the filter body 210 includes a bottom ring, a top ring, an inner wall an outer wall and a filter element. The inner wall and the outer wall are both annular. The inner diameter of the outer wall is larger than the inner diameter of the inner wall. The inner wall is fitted inside the outer wall and the inner and outer walls are coaxially arranged. There is an annular gap between the inner wall and the outer wall. The filter element is annular and is mounted at the annular gap to form a filter main body, and the bottom ring and the top ring are respectively mounted at upper and lower ends of the filter main body. The rotational snap-fit notches 211 are located at the lower edge of the inner wall, the limiting bosses 40 are located at the upper edge or the lower edge of the outer wall, and the handle 250 is located at a protruding structure formed by the limiting extension of the top ring.

Only a preferred embodiment of the present invention is described above, and the scope of protection of the present invention is not limited thereto. Any changes or substitutions that can be readily conceived by those skilled in the art within the technical scope disclosed by the present invention shall fall within the scope of protection of the present invention. Therefore, the scope of protection of the present invention should be subject, to the scope of protection of the claims.

1. A purifier, comprising a purifier body and a filter unit arranged in a purification chamber of the purifier body, wherein
a side wall of the purification chamber has an opening to which a cover is mounted;
the filter unit is an integrated filter and disinfection body, which comprises a filter body and a disinfection body which are detachable from each other; and
the filter unit is, mounted inside the purification chamber and electrically connected to the purifier body to supply power to the disinfection body.

2. The purifier of claim 1, wherein the filter unit is cylindrical, and an upper end of the filter unit has an opening as a purified air outlet, which is in communication with an air discharge passage of the purifier body; and
the disinfection body and the filter body are arranged one inside the other, and the filter body is arranged close to and in communication with an air intake passage of the purifier body.

3. The purifier of claim 1, wherein the disinfection body comprises an ultraviolet disinfection lamp and a mounting seat, wherein
the ultraviolet disinfection lamp is vertically mounted on the mounting seat, and is electrically connected to a first electrical connector arranged on the mounting seat; and
the mounting seat is a base seat having a bottom surface provided with the first electrical connector, and the first electrical connector is electrically connected to a second connector arranged on the purifier body.

4. The purifier of claim 1, wherein the filter body is cylindrical, the disinfection body is placed in the middle of the filter body, an inner side wall of the filter body is provided with at least one rotational snap-fit notch, and an outer side wall of the disinfection body is provided with at least one rotational snap-fit catch; and
the at least one rotational snap-fit catch is snap-fitted with or detached from the at least one rotational snap-fit notch by rotating the disinfection body relative to the filter body.

5. The purifier of claim 4, wherein there are a plurality of the rotational snap-fit notches, which are located at a lower edge of the filter body and arranged at intervals; and
the number of the rotational snap-fit catches matches the number of the rotational snap-fit notches.

6. The purifier of claim 1, wherein the filter unit is provided with at least one limiting boss, and the purification chamber is provided with at least one limiting column; and
the at least one limiting boss is mated and connected to the at least one limiting column to form a mounting limiting structure of the filter unit.

7. The purifier of claim 6, wherein there are four of the limiting bosses, which are divided into two groups each comprising two limiting bosses, the two groups of limiting bosses are symmetrically arranged, and the two limiting bosses in each group are arranged one above the other; and
there are two of the limiting columns, which match the two groups of limiting bosses.

8. The purifier of claim 7, wherein the two limiting bosses in each group are respectively located at upper and lower edges of the filter body.

9. The purifier of claim 1, wherein the filter unit is provided with a handle, which is located at an upper edge of the filter body.

10. The purifier of claim 1, wherein the side wall of the purification chamber or/and the cover is/are provided with air inlet holes.

11. The purifier of claim 2, wherein the disinfection body comprises an ultraviolet disinfection lamp and a mounting seat, wherein
the ultraviolet disinfection lamp is vertically mounted on the mounting seat, and is electrically connected to a first electrical connector arranged on the mounting seat; and
the mounting seat is a base seat having a bottom surface provided with the first electrical connector, and the first electrical connector is electrically connected to a second connector arranged on the purifier body.

12. The purifier of claim 2, wherein the filter body is cylindrical, the disinfection body is placed in the middle of the filter body, an inner side wall of the filter body is provided with at least one rotational snap-fit notch, and an outer side wall of the disinfection body is provided with at least one rotational snap-fit catch; and
the at least one rotational snap-fit catch is snap-fitted with or detached from the at least one rotational snap-fit notch by rotating the disinfection body relative to the filter body.
